(19) United States
(12) Patent Application Publication
(10) Pub. No.: US 2013/0028868 A1
(43) Pub. Date: Jan. 31, 2013

(54) CLINICAL APPLICATIONS OF A RECOMBINANT HUMAN ENDOSTATIN ADENOVIRUS (E10A) INJECTION

(71) Applicant: Wenlin Huang, Chadds Ford, PA (US)

(72) Inventor: Wenlin Huang, Chadds Ford, PA (US)

(21) Appl. No.: 13/549,629

(22) Filed: Jul. 16, 2012

Related U.S. Application Data
(60) Provisional application No. 61/509,228, filed on Jul. 19, 2011.
(60) Provisional application No. 61/509,231, filed on Jul. 19, 2011.

(51) Int. Cl. A61K 48/00; A61P 35/00; A61P 9/00; C12N 15/63

(52) U.S. Cl. 424/93.2; 435/320.1

(57) ABSTRACT

The present invention relates to the genetic engineering field of angiogenesis-targeted therapy. Particularly, a recombinant human endostatin adenovirus injection is used along with certain antitumor agents for treating specific tumors. The methods of the invention offer significant improvement of effective response rate, disease control rate, survival and quality of life. The injection and treatment has no significant adverse reactions and side effects.

### PRIORITY CLAIMS

This application claims the benefit of priority from U.S. Provisional Application Ser. Nos. 61/509,228, filed Jul. 19, 2011, and 61/509,231, filed Jul. 19, 2011, the entire content of each of which is incorporated herein by reference in its entirety.

### TECHNICAL FIELD OF THE INVENTION

The invention generally relates to compositions of genetically-engineered anti-angiogenesis agents and methods thereof for cancer treatment. More particularly, the invention relates to genetically-engineered (recombinant) human endostatin adenovirus (or other vector not limited to adenovirus) as cancer therapeutic in combination with other antitumor agents such as chemotherapeutics for treating specific tumors.

### BACKGROUND OF THE INVENTION

Angiogenesis is the physiological process involving the growth of new blood vessels from pre-existing vessels. (See e.g., John S. Penn (Mar. 11, 2008) Retinal and Choroidal Angiogenesis Springer, ISBN 9781402067792.) Tumors induce blood vessel growth (angiogenesis) by secreting various growth factors. Angiogenesis is a fundamental step in the transition of tumors from a dormant state to a malignant one. Angiogenesis is required for the spread of a tumor, or metastasis. Endothelial cells are reported to be genetically more stable than cancer cells, which stability confers an advantage to targeting endothelial cells using antiangiogenic therapy. Unlike chemotherapy drugs directed at cancer cells, which rapidly mutate and acquire “drug resistance” to treatment, endothelial cells are thought to be good targets for therapies.

Angiogenesis is a complicated, multistep process which includes degradation of the basement membrane, vascular endothelial cell proliferation, migration and differentiation. The process ultimately forms new blood vessels to provide oxygen and nutrition to the surrounding tissue. New blood vessel formation is determined by the balance between angiogenesis stimulatory factors with inhibitory factors.

Endostatin is a naturally occurring antiangiogenic protein that is reported to inhibit the formation of blood vessels that feed tumors. It was first discovered in the Children's Hospital Boston laboratory of Judah Folkman. As an endogenous angiogenesis inhibitor, endostatin may interfere with the pro-angiogenic action of growth factors such as basic fibroblast growth factor (bFGF/FGF-2) and vascular endothelial growth factor (VEGF). (Folkman, J. (2006) Exp. Cell. Res. 312 (5): 594-607.)

Endostatin was first found secreted in the media of non-metastasizing mouse cells from a hemangioendothelioma cell line and was subsequently found in human. Endostatin was reported to play a role in extracellular matrix in suppression of neoangiogenesis. (O'Reilly, et al. (1997) Cell 88: 277-85; Standker, et al. (1997) FEBS Lett. 420: 129-33.)

Endostatin has been identified as a C-terminal fragment of Collagen type 18. Endostatin has a short half-life and its therapeutic effect is in dose-dependent manner. Without wishing to be bound by the theories, endostatin represses cell cycle control and anti-apoptosis genes in proliferating endothelial cells, resulting in cell death. (Shichiri, et al. (2001). FASEB J 15: 1044-53.) Endostatin blocks pro-angiogenic gene expression controlled by c-Jun N terminal kinase (JNK) by interfering with TNFα activation of JNK. (Yin, et al. (2002) Mol. Ther. 5: 547-54.) It reduces the growth of new cells by inhibiting cyclin D1. As a result, cells arrest during G1 phase and enter apoptosis. (Dhanabal, et al. (1999) Biochem Biophys Res. Comm. 258: 345-52; Hanai, et al. (2002) J. Biol. Chem. 277: 16464-9.)

Endostatin has been reported to have several advantages in its use for cancer therapy. Endogenous endostatin has shown little or no resistance or toxicity in human compared to other cancer drugs. Endostatin has been estimated to affect about 12% of the human genome and offers a broad spectrum of potential activity as compared to single-molecule therapies.

There is a continued unmet need for safe and effective cancer therapies against various forms of cancer. Further development of endostatin-based cancer therapies may potentially provide revolutionary cancer treatments.

### SUMMARY OF THE INVENTION

The present invention is based, in part, on the novel approach to cancer treatment toward specific tumors that employ recombinant human endostatin adenovirus in combination with chemical antitumor drugs.

In one aspect, the invention generally relates to a recombinant human endostatin adenovirus composition for treating cancer. The composition includes: a therapeutically effective dosage of recombinant human endostatin adenovirus, wherein the composition is suitable for injective administration to a subject in need thereof and wherein the composition is suitable for co-administration with and alleviates toxic side effect of chemotherapy agents while substantially improving the overall effectiveness of treatment of cancer.

In certain preferred embodiments, the human endostatin genes are cloned from human fetal liver cells.

In certain preferred embodiments, the recombinant human endostatin adenovirus comprises a type V adenovirus gene and human endostatin gene.

In certain preferred embodiments, the cancer is selected from the group consisting of advanced head and neck malignant tumor, soft tissue sarcoma, and advanced breast cancer.

In certain preferred embodiments, the chemotherapy agents are selected from Paclitaxel and Cisplatin.

In certain preferred embodiments, the composition has a unit dosage amount of 1.0×10¹² VP.

In certain preferred embodiments, the unit dosage of Paclitaxel is 160 mg/m² on the third day and the unit dosage of Cisplatin is 25 mg/m² on the third day, the fourth day, and the fifth day of administration of the recombinant human endostatin adenovirus composition.

In another aspect, the invention generally relates to a method for treating cancer. The method includes: administering to a subject in need thereof a therapeutically effective amount of recombinant human endostatin adenovirus via a single or multi-point local injective administration applied to a tumor having a size of about 2 cm or greater given once weekly for 2 consecutive weeks as follows: on the 1st day a medication cycle, a 7-day interval, and a follow-up injection on the eighth day toward the tumor site.

In certain preferred embodiments, each injection has a unit dosage of 1.0×10¹² VP of recombinant human endostatin adenovirus.

In certain preferred embodiments, the method further includes administering Paclitaxel at 160 mg/m² (first-time dosage permitted for adjusting of not exceeding 10%) on the third day; or Cisplatin 25 mg/m² on the third day, the fourth day, and the fifth day.

### BRIEF DESCRIPTION OF THE DRAWINGS

FIG. 1 shows certain data collected in connection with Case #038: Metastatic nasopharyngeal carcinoma in neck lymph node; head and neck CT images before and after treatment with E10A according to an embodiment of the invention.

FIG. 2 shows certain data collected in connection with Case #038: Metastatic nasopharyngeal carcinoma in neck lymph node; right-side neck photos before and after treatment with E10A according to an embodiment of the invention.

FIG. 3 shows certain data collected in connection with Case #038: Metastatic nasopharyngeal carcinoma in neck lymph node; CT imaging of pulmonary metastasis before and after treatment with E10A according to an embodiment of the invention.

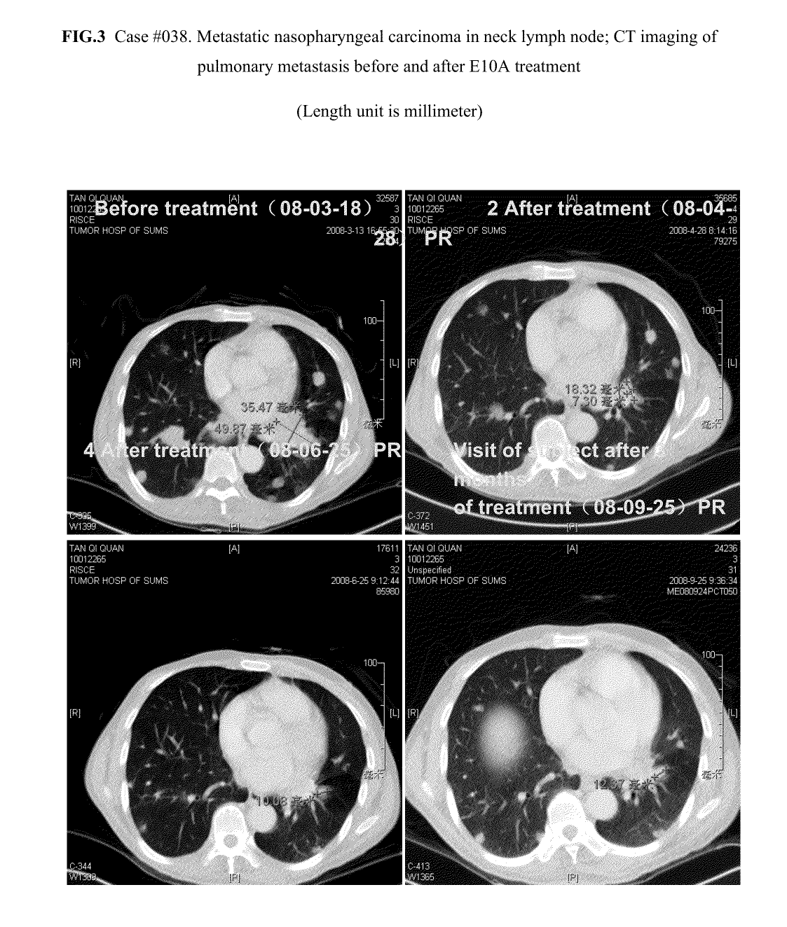

FIG. 4 shows certain data collected in connection with Case #042: Squamous-cell carcinoma of the left upper jaw; head and neck CT images before and after treatment with E10A according to an embodiment of the invention.

FIG. 5 shows certain data collected in connection with Case #042: Squamous-cell carcinoma of the left upper jaw; rear and front left upper jaw photos before and after treatment with E10A according to an embodiment of the invention.

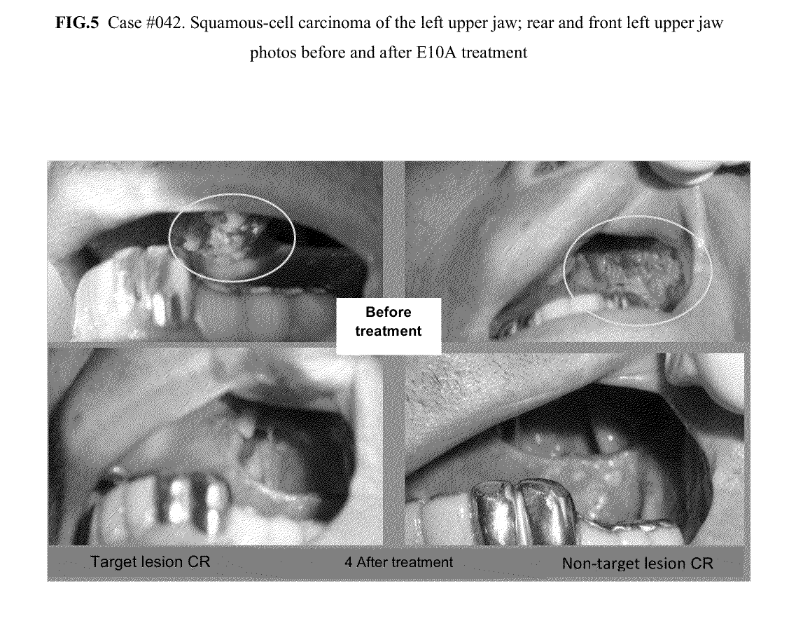

FIG. 6 shows certain data collected in connection with Case #069: Left tonsil cancer left common carotid artery sheath lymph node metastasis; head and neck CT images before and after treatment with E10A according to an embodiment of the invention.

FIG. 7 shows certain data collected in connection with Case #094: Nasopharyngeal carcinoma left common carotid lymph node metastasis; head and neck CT images before and after treatment with E10A according to an embodiment of the invention.

### DETAILED DESCRIPTION OF THE INVENTION

The present invention provides a novel approach to cancer treatment toward specific tumors that employs recombinant human endostatin adenovirus in combination with chemical antitumor drugs such as Paclitaxel/Cisplatin.

Advanced head and neck malignant tumor, soft tissue sarcoma, advanced breast cancer and other vascular-rich tumors are serious health threats to patients with poor prognosis. Currently, advanced surgeries apply pure chemotherapy or a combination therapy. Although these treatments have certain benefits, typical survival rates for such patients are quite low. The present invention offers a novel combination therapy using recombinant human endostatin adenovirus along with chemical antitumor drugs and opens a new path in tumor treatment for specific tumors.

Human endostatin is an angiogenesis inhibitor that inhibits the proliferation of vascular endothelial cells to achieve its purpose of inhibiting angiogenesis.

Adenoviruses are viruses that carry their genetic material in the form of double-stranded DNA. When adenoviruses infect a host cell, they introduce their DNA molecule into the host. The genetic material of the adenoviruses is not incorporated into the host cell's genetic material. The DNA molecule is left free in the nucleus of the host cell, and the instructions in this extra DNA molecule are transcribed just like any other gene. The only difference is that these extra genes are not replicated when the cell is about to undergo cell division so the descendants of that cell will not have the extra gene. As a result, treatment with the adenovirus will require re-administration in a growing cell population.

Here, the recombinant human endostatin adenovirus refers to the introduction of an improved type V adenovirus gene into human endostatin genes. The recombinant adenovirus serves as the vector to introduce human endostatin genes into the host cell gene structure, and yields human endostatin protein via gene expression. Human endostatin adenovirus of the present invention is a replication-defective, recombinant oncolytic adenovirus encoding human endostatin with potential antineoplastic activity. Upon intratumoral administration, the adenovirus infects and replicates in tumor cells. The expressed endostatin may inhibit endothelial cell proliferation and angiogenesis which may result in a reduction of tumor growth. The human endostatin genes used in the present invention are cloned from fetal liver cells.

In this present invention, other chemical antitumor drugs that may be used in combination with recombinant human endostatin adenovirus include Paclitaxel (See, Paclitaxel Product Insert, http://packageinserts.bms.com/pi/pi_taxol.pdf, accessed on Apr. 24, 2011) and/or Cisplatin. (See, Cisplatin Product Insert, http://www.bedfordlabs.com/products/inserts/CIS-AQ-P01.pdf, accessed on Apr. 24, 2011.) Specific tumors that may be effective treated include advanced head and neck malignant tumor, soft tissue sarcoma, advanced breast cancer and other vascular-rich tumor patients.

The recombinant human endostatin adenovirus injection uses the type V adenovirus, which introduces the human endostatin gene directly to the local tumor of a patient. Based on the transcription and translation mechanisms of the host cell, a continual expression (e.g., one-week) of therapeutic protein is provided to effectively inhibit the formation of new vessels within the tumor tissue, resulting in apoptosis and necrosis of the cancer tissues

In this present invention, combination therapy refers to single or multi-point E10A local injection of recombinant human endostatin adenovirus (E10A) applied to tumor with sizes larger than 2 cm, with the said injection at dosage level of 1.0×10¹² VP, given once weekly for 2 consecutive weeks, having a medication cycle on the first day, a 7-day interval, and a follow-on injection on the eighth day toward the tumor site; the combination of Paclitaxel 160 mg/m² (first-time dosage permitted for adjusting of not exceeding 10%) and the third day medication implement the medication instruction manual (Paclitaxel product insert); or Cisplatin 25 mg/m², the third, fourth, and fifth day medication implement the medication instruction manual (Cisplatin product insert).

Random comparison study was conducted to compare the efficacy and safety of E10A-chemotherapy combination therapy with chemotherapy treatment alone for advanced head and neck squamous-cell carcinoma or nasopharyngeal carcinoma. Besides head and neck cancer or nasopharyngeal carcinoma, a self-control comparison was conducted to study the efficacy and safety of the E10A-chemotherapy combination therapy for advanced head and neck malignant tumor, soft tissue sarcoma and advanced breast cancer. Through rigorous experiment design, selection, exclusion criteria, medication planning, the observed disease control rate endpoint and efficacy response rate to the tumor lesion target were calculated (by the SAS9.3 statistical analysis software). The statistical tests of efficacy indicator ware done by one-tailed test, whereas the other indicators were based by the two-tailed test, α=0.05. The tests verified that the combination of recombinant human endostatin adenovirus injection with Paclitaxel/Cisplatin significantly improved the efficacy response rate and disease control rate for patients with advanced head and neck malignant tumor, soft tissue sarcoma, and advanced breast cancer. The combination therapy approach of the present invention exhibits a synergistic effect without any significant adverse drug reaction and toxic side effects.

The present invention applies the second-generation recombinant adenovirus (rAds) type V vector to introduce a temperature-sensitive mutation into the E2A gene for reducing the vector's immunogenicity. Immunohistochemistry detection indicated that the injection did not have replication and packaging capabilities after intratumoral injection. The in vivo treatment experiment demonstrated the safety of the gene therapy treatment with no significant side effects. The endostatin distribution experiment showed that the receiving of high-concentration endostatin expression and recombination of human endostatin adenovirus by intratumoral injection mainly took place at the localized tumor sites, whereas the expression levels in the plasma were lower. In addition, no apparent effects were observed among the organs of the entire body, reducing the prospect of toxic effects during treatment cycles of endostatin.

The improved type V adenovirus vector offers the advantages of producing high-titer recombinant adenovirus, boasting expression of the human endostatin gene in various cells, and increasing biological activity of the gene expression product. For in vitro experiments, the human endostatin recombinant adenovirus is used to infect vascular endothelial cells, and Western blot indicated that the endothelial cells have a continual human endostatin expression up to 7 days and more. Flow cytometry (FCM) indicated that after 24 hours of infection, the vascular endothelial cells appear G2 arrest, with large amounts of dead cell fragments occurring on the fourth day. The human endostatin recombinant adenovirus was used to infect human tongue cancer cells, Tca8113. The collected tongue cancer cell supernatant was measured for human endostatin concentration by enzyme-linked immunoassay (ELISA). It was shown that the human endostatin concentration of the infected cell supernatant increased as the multiplicity of infection (MOI) and infection period increased. At MOI=100, the peak concentration at 597.54 ng/mg was reached after 72 hours of the infection. The result indicates that the human endostatin recombinant adenovirus has a greater in vitro biological activity.

Cancer cell line in vivo experiments in nude mice were conducted for nasopharyngeal carcinoma (CNE-2 tumor cells) and liver cancer (BEL-7402 tumor cells). It was showed that the human endostatin recombinant adenovirus significantly inhibited the growth of subcutaneous tumor. Additionally, the serum human endostatin concentration within for the treatment group was significantly higher than the control group at seven days after the injection. This strongly indicated that the recombinant adenovirus carrying of human endostatin genes retains high-effectiveness in vivo. The extended therapeutic effect resulted from a long expression period which provided significantly inhibitory effect on the cancer cell line growth in nude mice.

In one aspect, the invention generally relates to a recombinant human endostatin adenovirus composition for treating cancer. The composition includes: a therapeutically effective dosage of recombinant human endostatin adenovirus, wherein the composition is suitable for injective administration to a subject in need thereof and wherein the composition is suitable for co-administration with and alleviates toxic side effect of chemotherapy agents while substantially improving the overall effectiveness of treating cancer.

In certain preferred embodiments, the human endostatin genes are cloned from human fetal liver cells.

In certain preferred embodiments, the recombinant human endostatin adenovirus comprises a type V adenovirus gene and human endostatin gene.

In certain preferred embodiments, the cancer is selected from the group consisting of advanced head and neck malignant tumor, soft tissue sarcoma, and advanced breast cancer.

In certain preferred embodiments, the chemotherapy agents are selected from Paclitaxel and Cisplatin.

In certain preferred embodiments, the composition having a unit dosage is 1.0×10¹² VP.

In certain preferred embodiments, the unit dosage of Paclitaxel is160 mg/m² on the third day and the unit dosage of Cisplatin is 25 mg/m² on the third day, the fourth day, and the fifth day of administration of the recombinant human endostatin adenovirus composition.

In another aspect, the invention generally relates to a method for treating cancer. The method includes: administering to a subject in need thereof a therapeutically effective amount of recombinant human endostatin adenovirus via a single or multi-point local injective administration applied to a tumor having a size of about 2 cm or greater given once weekly for 2 consecutive weeks as follows: with a medication cycle on the 1st day, a 7-day interval, and a follow-up injection on the eighth day toward the tumor site.

In certain preferred embodiments, each injection having a unit dosage of 1.0×10¹² VP of recombinant human endostatin adenovirus.

In certain preferred embodiments, the method further includes administering Paclitaxel at160 mg/m² (first-time dosage permitted for adjusting of not exceeding 10%) on the third day; or Cisplatin 25 mg/m² on the third day, the fourth day, and the fifth day.

### EXAMPLES

### 1. Advanced Head and Neck Squamous-Cell Carcinoma or Nasopharyngeal Carcinoma

Random comparison was used to compare the antitumor efficacy and safety of E10A combination chemotherapy with that of chemotherapy alone for advanced head and neck squamous-cell carcinoma or nasopharyngeal carcinoma.

The test and control groups were randomly selected to maintain a balanced patient baseline. The test group received E10A+Paclitaxel/Cisplatin chemotherapy, named as test group; the control group was based on pure Paclitaxel /Cisplatin chemotherapy, named as control group. Qualified subjects were randomly distributed to the two groups and received respective treatments. The efficacy response rate (PR/CR ratio) was used as the major observing endpoint. At the end, the efficacy response rate of the two groups was compared via differential analysis. The necessary sample size was estimated of not exceeding 58 cases for each group.

Patient Inclusion Criteria:

(1) Patient is unsuitable for surgery or radiation therapy for local advanced or metastatic head and neck cancer (head and neck squamous-cell carcinoma or nasopharyngeal carcinoma);

(2) Patient has at least one target lesion for proceeding intratumoral E10A injection;

(3) Patient has at least one tumor lesion with a measurable maximum size≧2 cm;

(4) Patient has a clear histological diagnosis;

(5) Patient has not received any antitumor treatments within 4 weeks, such as chemotherapy, radiation therapy or biological therapy;

(6) Patient is not less than 18 weeks old;

(7) Patient as an expected survival period≧12 weeks;

(8) Patient has an ECOG performance status of 0-2;

(9) Laboratory test results meet with the following condition one week prior to testing: Absolute neutrophil count (ANC)≧1.5×10⁹/L, number of platelets (PLT)≧80×109/L, hemoglobin (Hb)≧80 g/L; Total bilirubin (TBI)≦1.5×upper normal limit (2 mg/dl), Alanine transaminase (ALT), Aspartate aminotransferase (AST)≦2×upper normal limit; creatinine (Cr)≦1.5×upper normal limit; extension of activated partial thromboplastin period may not exceed 10 seconds of the APTT upper normal limit, and may not exceed three seconds of the PT upper normal limit.

(10) Voluntary participation by the patient with informed written consent.

Patient Exclusion Criteria:

(1) Patient is known to be allergic to a research agent;

(2) Patient's lesion consisting of important blood vessels and nerves that are unsuitable for local injection;

(3) Patient is receiving lesion radiation therapy;

(4) Patient had recurrence within six months after receiving Paclitaxel type chemotherapy treatment;

(5) Patient has serious clotting conditions or risks such as bleeding tendency;

(6) Patient has serious, uncontrolled medical conditions, history of recent myocardial infarction (within three months) or acute infection;

(7) Patient is pregnant or breast-feeding;

(8) Patient has symptoms of brain metastasis.

Treatments:

Test group Patients of this group received the combination of E10A injection with Paclitaxel/Cisplatin chemotherapy as follows: Single or multi-point E10A local injection applied to tumor with dosage level of 1.0×10¹² VP. Each medication cycle begins on the first day, a 7-day interval, and a follow-on injection on the eighth day toward the tumor site; the combination of Paclitaxel 160 mg/m² (first-time dosage permitted for adjusting of not exceeding 10%), medication is given on the third day and implemented by the medication instruction manual; or of a 25 mg/m² combination, which medication is given on the third day, the fourth day, and the fifth day in accordance to the medication instruction manual.

Control group Patients of this group received a standard Paclitaxel/Cisplatin chemotherapy as follows: Paclitaxel 160 mg/m² (first-time dosage permitted for adjusting of not exceeding 10%), medication on the first day and implemented by the medication instruction manual (See, Paclitaxel product insert); Cisplatin 25 mg/m², medication is given on the first day, the second day, and the third day in accordance to the medication instruction manual (See, Cisplatin product insert).

### 2. Advanced Head and Neck Malignant Tumor, Soft Tissue Sarcoma and Advanced Breast Cancer

Besides head and neck cancer or nasopharyngeal carcinoma, the efficacy and safety of E10A injection with Paclitaxel/Cisplatin chemotherapy treatment for advanced head and neck malignant tumor, soft tissue sarcoma and advanced breast cancer were observed.

Under the principle of not changing the original chemotherapy as basis, the self-controlled method was used to compare the lesion efficacy between the test group with E10A local injection and the control group without E10A injection.

Patient Inclusion Criteria:

(1) Patient is unsuitable for surgery or radiation therapy for local advanced or metastatic head and neck malignant tumor, soft tissue sarcoma or advanced breast cancer (advanced head and neck squamous-cell carcinoma or nasopharyngeal carcinoma);

(2) Patient has at least two target lesions for proceeding intratumoral E10A injection;

(3) Patient has at least one tumor lesion with a measurable (medical imaging or photo) maximum size≧2 cm;

(4) Patient has a clear histological diagnosis;

(5) Patient has not received any antitumor treatments within 4 weeks, such as chemotherapy, radiation therapy and biological therapy;

(6) Patient is not less than 18 weeks old;

(7) Patient has an expected survival period≧12 weeks;

(8) Patient has an ECOG performance status of 0-2;

(9) Laboratory test results meet with the following condition 1 week prior to testing: Absolute neutrophil count (ANC)≧1.5×10⁹/L, number of platelets (PLT)≧80×10⁹/L, hemoglobin (Hb)≧80g/L; Total bilirubin (TBI)≦1.5×upper normal limit (2 mg/dl), Alanine transaminase (ALT), Aspartate aminotransferase (AST)≦2×upper normal limit; creatinine (Cr)≦1.5×upper normal limit; extension of activated partial thromboplastin period may not exceed 10 seconds of the APTT upper normal limit, and may not exceed 3 seconds of the PT upper normal limit.

(10) Voluntary participation by the patient with informed written consent.

Patient Exclusion Criteria:

(1) Patient is known to be allergic to a research agent;

(2) Patient's lesion consisting of important blood vessels and nerves that are unsuitable for local injection;

(3) Patient is receiving lesion radiation therapy;

(4) Patient has serious clotting conditions or risks such as bleeding tendency;

(5) Patient has serious, uncontrolled medical conditions, history of recent myocardial infarction (within three months) or acute infection;

(6) Patient is pregnant or breast-feeding;

(7) Patient has symptoms of brain metastasis.

Treatments:

Based on the original chemotherapy plan, an additional E10A local injection of 1.0×10¹² VP is given on the first day and the eight day of the chemotherapy cycle.

Endpoint of Treatment:

Every 21 days is a treatment cycle. Unless the patient occurs with progress of the disease or intolerable toxic reaction, efficacy evaluation is conducted after two treatment cycles. Patients who reached partial remission were given at most six cycles of treatments, while stable disease (SD) patients received at most four cycles of treatments. The testing applies the best efficacy between the second and the fourth treatments as the main efficacy evaluation endpoint.

Efficacy Evaluation Endpoint:

Main endpoint Efficacy response rate of target tumor lesion (defined as PR/CR, partial remission /complete response ratio).

Secondary Endpoint

(1) Disease control rate efficacy of target tumor lesion, defined as total sum of stable disease (SD)+partial remission (PR)+complete response (CR);

(2) Efficacy response rate of total lesions;

(3) Disease exists with no progress and survival period;

(4) Remission phase;

(5) Immune response (Quantitative measurement of anti-adenovirus IgG antibodies in serum);

(6) Endostatin of gene expression (selective) safety evaluation: Including observations and recording all adverse and seriously adverse events, laboratory examination, general physical examination, ECoG evaluation, and ECG. Adverse events are classified in accordance with the toxicity scale, version 2.0, of the National Cancer Institute (USA) for Common Toxicity Criteria (NCI-CTC).

Statistical Analyses:

Test Group: The test and control groups are randomly selected to maintain a balanced patient baseline. Qualified subjects were randomly distributed to the two groups and received treatment. The efficacy response rate (PR/CR ratio) was used as the main observing endpoint. At the end, the efficacy response rates of the two groups were compared via differential analysis. The necessary sample size was estimated of not exceeding 58 cases for each group.

Control Group: Fourteen cases of various cancers are provided as basis for E10A treatment towards tumor cells.

Tests were conducted within the same period.

Statistical analyses were performed by the SAS9.3 statistical analysis software. The statistical tests of efficacy indicator were done with one-tailed test, while the other indicators were based by the two-tailed test, α=0.05. If the P value equals 0.05 or less, the difference was considered to be statistically meaningful.

Summary:

A total of 109 patients were selected from March 2008 to March 2010. The Test Group had 57 cases and the Control Group 52 cases, as listed in Table 1.

TABLE 1
Distribution of Cases and Completion Status

| | Test Group | Control Group | |
|---|---|---|---|
| | (E10A + Chemotherapy) | (Chemotherapy) | Total |
| Completed | 37 | 41 | 78 |
| In progress | 10 | 7 | 17 |
| Withdrew | 7 | 3 | 11 |
| Removed | 3 | 1 | 5 |
| Total | 57 | 52 | 109 |

With the exception of withdrawal and removed cases, the total of completed cases from March 2008 to March 2010 were 78 cases, of which 37 were of the Test Group and 41 in the Control Group, as listed in Table 2.

TABLE 2
General Information of Test Subjects

| | | Test Group | |
|---|---|---|---|
| | | (E10A + | Control Group |
| | | Chemotherapy) | (Chemotherapy) |
| Number of cases | | 37 | 41 |
| Age | | 23-69 | 30-74 |
| Gender | Male | 28 | 30 |
| | Female | 9 | 11 |
| Diagnosis | Head and neck | 21 | 21 |
| | cancer | | |
| | Nasopharyngeal | 16 | 20 |
| | carcinoma | | |

Note:
Statistical analysis was conducted toward completed cases (completed with at least 2 treatments, and conducted 2 efficacy evaluations)

The efficacy response rate, disease control rate and total efficacy response rate of the Test group and Control Group were analyzed by χ² testing or continuous calibrated χ² testing. The difference between the two groups were both at significant results (P<0.05), with the results listed in Table 3.

TABLE 3
Analysis of Efficacy Response Rate
and Control Rate to Target Lesion

| | | CR | PR | SD | PD | Total |
|---|---|---|---|---|---|---|
| | | N (%) | N (%) | N (%) | N (%) | N |
| Test | Target | 2 (5.4) | 16 (43.2) | 13 (35.1) | 6 (16.2) | 37 |
| Group | Lesion | | | | | |
| | Overall | 1 (2.7) | 15 (40.5) | 11 (29.7) | 10 (27.1) | 37 |
| Control | Target | 0 (0) | 10 (24.4) | 16 (39.0) | 15 (36.6) | 41 |
| Group | Lesion | | | | | |
| | Overall | 0 (0) | 9 (22.0) | 14 (34.1) | 18 (43.9) | 41 |

Note:
The target lesion efficacy response rate (CR + PR/CR + PR + SD + PD) χ² testing: χ² = 3.282, P = 0.0257;
Target lesion disease control rate (CR + PR + SD/CR + PR + SD + PD) χ² testing: χ² = 3.282, P = 0.043;
Overall efficacy response rate χ² testing: χ² = 3.282, P = 0.044.

Exemplary/Typical Cases:

1. Case #038

Subject: Male, age 55. Clinical diagnosis was “nasopharyngeal carcinoma recurrence after radiotherapy; neck lymph node and pulmonary metastasis”.

Target Lesion: Right auxiliary lymph node.

Treatment Process: Patient was randomly selected into the Test Group and was given four treatments of E10A+Paclitaxel (TP) chemotherapy on Mar. 19, 2008, Apr. 9, 2008, Apr. 30, 2008, and May 21, 2008, respectively.

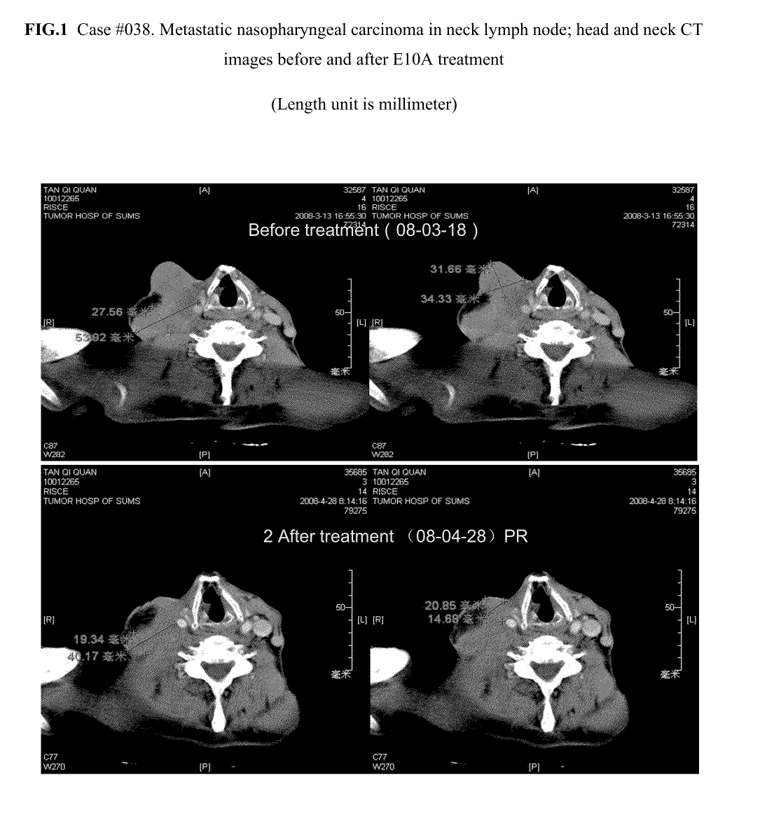

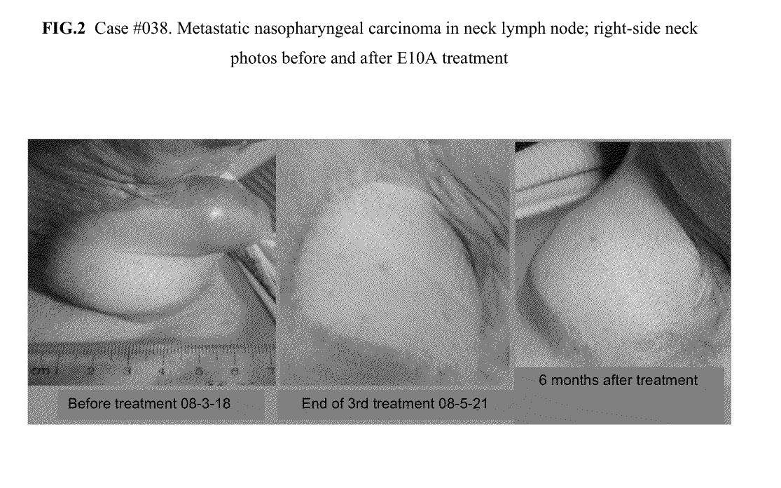

Tumor Evaluation: After two treatments (Apr. 4, 2008), efficacy evaluation of the target lesion was PR, and non-target lesion was PR; after four treatments (Jun. 25, 2008), target lesion efficacy was CR, non-target lesion efficacy was PR, and overall efficacy was PR. (FIGS. 1, 2 & 3).

Follow-up: A follow-up CT examination was performed on Sep. 27, 2009, which showed size increase of pulmonary lesion and progressive disease (PD) condition.

2. Case #042

Subject: Male, age 61. Clinical diagnosis was “squamous-cell carcinoma of the left upper jaw”.

Target Lesion: Left upper jaw mass; non-target lesion: left jaw.

Treatment Process: Patient was randomly selected into the Test Group and was given four treatments of E10A+Paclitaxel (TP) chemotherapy on Jul. 10, 2008, Jul. 31, 2008, Aug. 21, 2008 and Sep. 11, 2008, respectively.

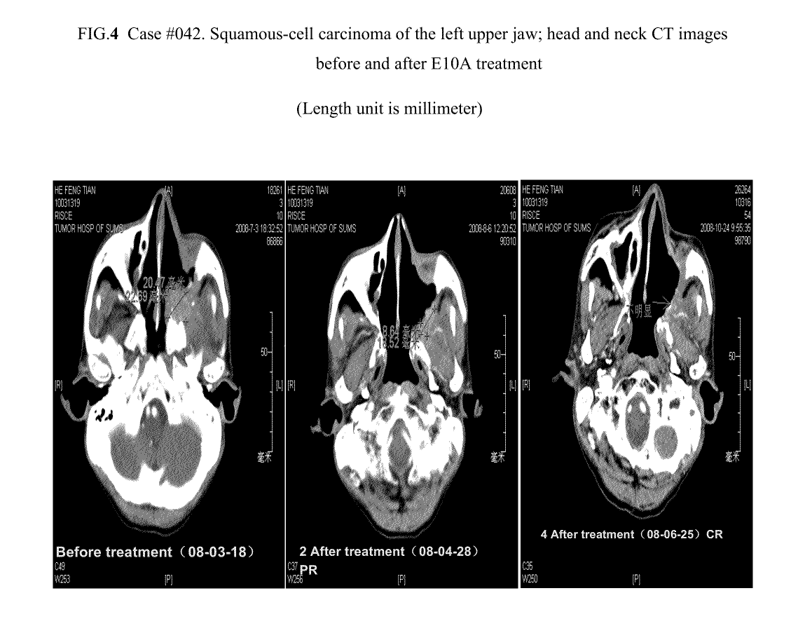

Tumor Evaluation: After two treatments (Aug. 6, 2008), efficacy evaluation of the target lesion was PR, and non-target lesion was PR. After four treatments (Oct. 24, 2008), target lesion efficacy was CR, non-target lesion efficacy was CR, and overall efficacy was CR. (FIGS. 4 & 5)

Follow-up: A follow-up examination was conducted on Oct. 30, 2008. The right upper jaw tumor mass disappeared, ulcer of left tonsil disappeared. Cleaning was performed. Condition continued as CR until May 8, 2009. CT imaging on May 12, 2009 showed left edge of the surgical nodules with high possibility of new lesion. Return visit on Jul. 27, 2009 indicated left palate surgical site appear with cauliflower-like mass, with size approximately 1.5 cm×2 cm, medium texture and border still clear.

3. Case #069

Subject: Male, age 44. Clinical diagnosis was “left tonsil cancer left common carotid artery shealth lymph node metastasis”.

Target Lesion: Left common carotid artery sheath lymph node.

Treatment Process: Patient was randomly selected into the Test Group and was given four treatments of E10A+Paclitaxel (TP) chemotherapy on Feb. 13, 2009, Mar. 6, 2009, Mar. 27, 2009, and Apr. 17, 2009, respectively.

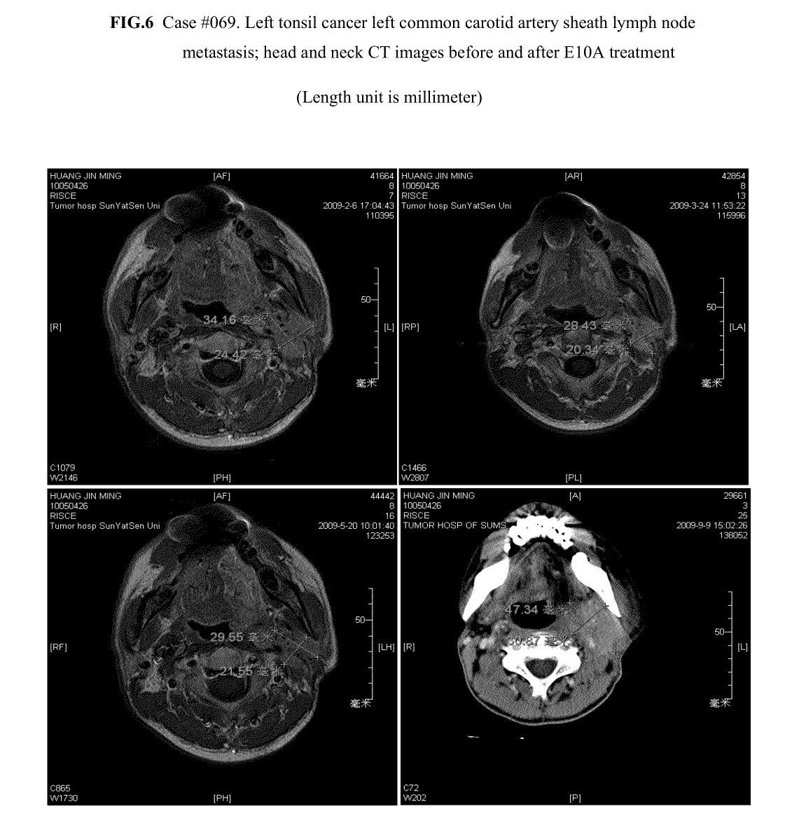

Tumor Evaluation: After two treatments (Mar. 24, 2009), efficacy evaluation of the target lesion was SD. After four treatments (May 20, 2009), target lesion efficacy was PR. (FIG. 6)

Follow-up: Patient described of headaches occurring at the left side starting 20 days ago, frequent coughing when drinking or eating, weight loss of 4 kg, experienced blurred vision twice, cold sweat and fainting symptoms.

4. Case #094

Subject: Female, age 42. Clinical diagnosis was “nasopharyngeal carcinoma left common carotid lymph node metastasis”.

Target Lesion: Left cervical lymph node; non-target lesion: right cervical lymph node.

Treatment Process: Patient was randomly selected into the Test Group. The patient was given four treatments of E10A+Paclitaxel (TP) chemotherapy on May 27, 2009, Jun. 17, 2009, Jul. 8, 2009, and Jul. 29, 2009, respectively.

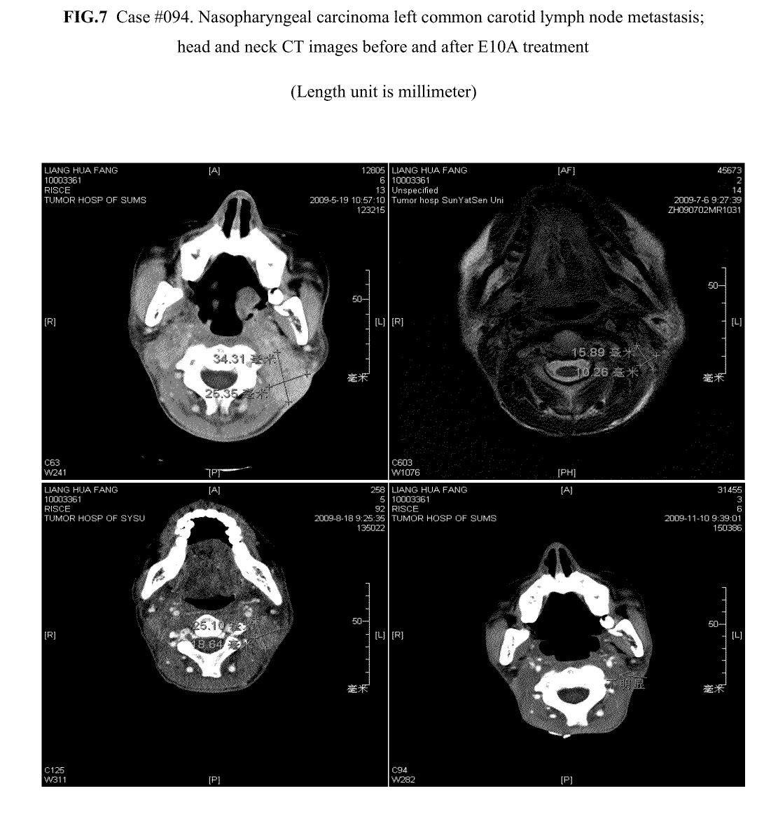

Tumor Evaluation: After two treatments (Jul. 6, 2009), efficacy evaluation of the target lesion was PR, non-target lesion was PR. After four treatments (Aug. 18, 2009), target lesion efficacy was PR, and non-target lesion was PR. (FIG. 7)

Follow-up: CT imaging on Nov. 10, 2009 indicated that the tumor size significantly shrank.

In cases of multiple lesions, the dosages were based on the amount of tumor lesions, and a rotated medication was applied: For example 3-4 tumors were based as one group, which after medication of the first group, medication for the second group was given on the fourth day, and medication for the third group was given three days after the second-group medication, with a medication cycle of 10 days.

Treatment Safety:

Adverse Events: No detection of drug-related serious adverse events. Major adverse drug reactions were local reaction and febricity.

Local Reaction: No serious reactions were found. Slight local pain or swelling (20%) may occur after intratumoral injection of E10A.

Febricity: After intratumoral injection with E10A, 45% of subjects occurred febricity with highest temperatures at 37.2° C.-40.1° C., continuing between 2-24 hours, accompanied with different levels of increased heart beat rate and transient blood pressure changes. Subjects were able to ease febricity by their own, or were quickly alleviated after giving acetaminophen or physical cooling.

Other Adverse Drug Reactions: Bone marrow inhibition, gastrointestinal reactions and other adverse drug reactions such as hair loss and allergy. Incidences of adverse drug reactions were quite the same between the Control Group and Test Group.

These examples demonstrated that the combination therapy of recombinant human endostatin adenovirus injection in combination with Paclitaxel /Cisplatin can significantly improve the efficacy response rate and disease control rate of advanced head and neck malignant tumor, soft tissue sarcoma, advanced breast cancer and other vascular-rich tumor patients. In many cases, the tumors shrank in size or even disappeared, and the overall survival rate and quality of life were significantly enhanced.

Thus, the combination therapy approaches of the present invention offer a synergistic effect without any significant adverse drug reaction or toxic side effects during the chemotherapy period. The recombinant human endostatin adenovirus injection and Paclitaxel/Cisplatin combination treatment is a safe and effective treatment method for patients with advanced head and neck malignant tumor, soft tissue sarcoma, advanced breast cancer, and other vascular-rich tumor patients.

### APPENDIX

1. Medication Instruction Manual—Paclitaxel

[Name of drug]: Paclitaxel Injection

[Chemical name]: 5β, 20-epoxy-1, 2α, 4, 7α, 1-β, 10α-hexahydroxytax-11-en-9-one-4,10-diacetate-2-benzoate-13-[(2′R, 3′S)—N-benzoate-3-phenylisoserine

[Molecular formula]: C₄₇H₅₁NO₁₄

[Molecular weight]: 853.92. Drug class: Chemotherapy drug, plant alkaloid

[Appearance]: Colorless or slight yellow transparent, viscous liquid.

[Pharmacology & Toxicology]:

Paclitaxel is a new anti-microtubule drug, which promotes tubulin polymerization, inhibits depolymerization to maintain tubulin stability and inhibit cell mitosis. In vitro experiments proved Paclitaxel has significant radiosensitization, which perhaps contribute to ceasing the radiosensitive G2 and M phases for cells.

[Pharmacodynamics]:

Paclitaxel is a mitotic inhibitor or spindle poison, having a different mechanism than the commonly used chemotherapy drugs. It inducts and promotes microtubule assembly. Paclitaxel is shown to bind to and stabilize microtubules, causing the rapid dividing of tumor cells to be securely fixed in the mitotic phase. This results the blocking of cancer cell replication, leading to cancer cell death.

[Pharmacokinetics]:

The drug concentration-time curve for intravenous paclitaxel shows of a two-compartment model. Protein binding is 89%-98%. Paclitaxel metabolism takes place mainly in the liver, which enters the intestine along with bile and excreted with feces (>90%). Renal clearance of the drug only occupies 1-8%. The metabolism of Paclitaxel for liver and kidney dysfunction patients is still is not clear.

[Indications]:

First and second line treatment for ovarian cancer, breast cancer and non-small cell lung cancer (NSCLC). Shows certain efficacy toward head and neck cancer, esophageal cancer, germ cell tumor, and recurrent Non-Hodgkin's Lymphoma.

### Indications:

Paclitaxel injection is a broad spectrum antitumor drug:

1, One of the most effective drug for treating refractory ovarian cancer;

2, Has an important role in breast cancer treatment;

Indications expansions of the drug are currently applied to the following:

1, Combination therapy for lung cancer;

2, For the treatment toward malignant head and neck squamous-cell carcinoma;

3, Applied in clinical tests toward other malignant tumors.

[Dosage]:

To prevent the occurrence of allergy with the patient, the patient should be first given preventive medication, which dexamethasone oral is given 12 hours and 6 hours, respectively, in advance; diphenhydramine 50 mg intramuscular injection (or oral), Cimetidine 300 mg intravenous injection or Ranitidine 50 mg is given 30 minutes before administration.

The drug is diluted in 500 ml of 0.9% sodium chloride injection or 5% glucose injection before clinical use. Intravenous infusion is done with a non-PVC infusion bottle and infusion tube, and filtered by passing through a connected screening program (0.22 μm pore size).

The dosage is calculated based on 175 mg/m2 of body surface area, 3 hours of constant infusion once for every 3 weeks. The dosage amount and number of times can be adjusted based on the situation.

The blood pressure, heart rate and breathing frequency are measured every 15 minutes after 1 hour since treatment. Notice for allergy.

[Adverse Drug Reaction]:

1. Allergy: Rate of incidence 39%, which serious allergy incidence is 2%. Most are of type I hypersensitivity. Symptoms are bronchospasm, dyspnea, urticaria and low blood pressure. Almost all reactions occur at 10 minutes of the first post-treatment.

2. Marrow inhibition: The main dose limiting toxicity, manifested with decreased neutrophil cells, reduction of platelets and rare amount, usually occurring 8-10 after medication. Incidence of serious neutrophil cell reduction is 47%, and serious platelet reduction is 5%. Anemia is commonly seen.

3. Neurotoxicity: Incidence of peripheral neuropathy at 62%. Incidence rate of serious neurotoxicity is 6%.

4. Cardiovascular toxicity: May occur low blood pressure without symptoms of short time bradycardia, and 30% having ECG abnormalities.

5. Muscle and joint pain: Incidence at 55%, occurring in the limbs and joints. Incidence and serious levels appear as dose-dependent reactions.

6. Gastrointestinal: Incidence of nausea, vomiting and diarrhea mucositis respectively at 59%, 43% and 39%. Usually are mild and moderate.

7. Liver toxicity: Increase of alanine transaminase (ALT) , aspartate aminotransferase (AST) and alkaline phosphatase (AKP).

8. Hair loss: 80% incidence.

9. Local reaction: Intravenous infusion of drug and local inflammation of drug extravasation. of.

[Notice]:

1. To prevent possible allergy, Dexamethasone, Diphenhydramine and H2 receptor antagonist pretreatment should be administrated prior to Paclitaxel treatment.

2. Precaution required with Paclitaxel preparation. Wear gloves. If contact with skin, immediately use soap to wash the infected skin thoroughly. Wash thoroughly wash with water if contact with mucous membrane.

3. Intravenous injection is to be stopped immediately when extravascular leakage occurs. Apply local cold compress and 1% procaine.

4. The blood pressure, heart rate and breathing frequency are measured every 15 minutes after 1 hour since treatment. Notice for allergy.

5. Paclitaxel infusion should be implemented with non-PVC infusion bottles and infusion devices. The process should be connected to a screening program, which the micro-pores of the filter membrane should be less than 0.22 micron.

6. Concentrated Paclitaxel injection is required to be diluted before intravenous injection, using physiological saline, 5% glucose or 5% glucose saline. The final diluted concentration is 0.3˜1.2 mg/ml.

7. Usage of the drug should be guided by an experienced tumor chemotherapy physician. The patient must be hospitalized, and prepared with anti-allergy drugs and appropriate rescue equipment prior to injection.

[Medication for Pregnant and Lactating Women]:

Paclitaxel has confirmed to influence embryonic growth in animal experiments, therefore pregnant and lactating women are prohibited of use. Women should not become pregnant during treatment.

[Drug Interactions]:

1. Quinupristin/Dalfopristin serves as an inhibitor to cell pigment P450-3A4. At the same time, it can increase plasma concentration.

2. When used together with Trastuzumab, the serum alanine concentration level increases about 1.5 times. Clinical tests proved that the combination provides a better effect.

3. Cisplatin reduces clearance of the drug about ⅓. If Cisplatin is used before drug administration, an even more severe inhibition of bone marrow shall occur.

4. When combined with Adriamycin, studies show that when 24 hours continuous infusion of the drug is applied before giving 48 hours continuous infusion of Adriamycin can significantly lower the rate of Adriamycin clearance, increase neutrophil cell reduction and stomatitis.

5. Adriamycin immediately given after drug usage can increase drug toxicity.

6. Ketoconazole may inhibit drug metabolism.

7. Phosphorous phenytoin and phenytoin can reduce drug performance by induction of cell pigment P450.

8. Inoculation of live vaccine (such as Rotavirus vaccine) while using the drug may increase the risk of vaccine infection. Data from abroad reports suggest that when using this drug, live vaccine inoculation is to be prohibited. Patients under white blood cell remission phase and patients ended with chemotherapy should wait at least three months before receiving live vaccine inoculation.

[Contraindications]:

1. Persons who are allergic to the drug, or to other polyoxyethylene castor preparations

2. White cell count lower than 1.5×109/L, serious marrow inhibition.

3. Pregnant and lactating women.

[Drug Overdose]:

There is presently no corresponding antidote known. Major and predictable complications with drug overdose include marrow inhibition, peripheral neurotoxicity and mucositis.

[Specification]: 5 ml 30 mg.

[Storage]: Avoid light. Store in a sealed container under 25° C.

[Valid Period]: Two years

### 2. Medication Instruction Manual—Cisplatin

[Name of drug]: Cisplatin Injection

[Name of English]: cisplatin Injection

[Major substance]: Cisplatin

[Appearance]: Transparent, slight viscous liquid with a slight yellow color.

[Pharmacology & Toxicology]:

Reproductive toxicity: Application of this product to pregnant women may cause fetal harm. Murine tests show teratogenicity and embryo toxicity. If used during pregnancy, or found to be pregnant during administration, the patient should be informed of the potential harm to fetus. Woman tend to childbearing should be advised to avoid pregnancy. Reports indicated of drug detected in human milk, therefore the mother should stop breastfeeding while using this product.

Genetic toxicity: The drug shows mutagenicity in bacterial tests, causing chromosome aberration to animal tissue culture.

Carcinogenicity: Carcinogenicity test in BDIX rats (50 rats) resulted in 33 deaths within 455 days after first time administration, giving medication of 3 weeks, 1 mg/kg/week. Among them 13 died from malignant tumor, 12 from leukemia, and 1 from renal fibrosarcoma.

[Pharmacodynamics]:

Pharmacodynamics of the drug is of metal platinum complex, cycle phase-non-specific antitumor, demonstrated of broad-spectrum antitumor and effective to hypoxic cells. The drug rapidly disassociates in low chloride cell environments, combining to large molecules within the cell as hydrated cations (mainly targeting DNA) to form chains, chain linkage, cross-linked protein or DNA cross-linkage. This destructs the DNA structure and function. It is believed that the main reaction takes place at the purine and pyrimidine base of DNA.

[Pharmacokinetics]:

Rapidly absorbed through intravenous, intraluminal or arterial injection. The drug broadly distributes to the liver, kidney, prostate, bladder, ovary, and also the chest and abdominal cavity after injection. Only a very small amount passes through the blood-brain barrier; T1/22 and above. When combined with diuretics T1/2 significantly shortens. The drug is mainly excreted by renal excretion, passing through glomerular filtration or in a part by tubular secretion. About 25%˜45% is excreted along with urine 96 hours after treatment. After injection into the abdominal cavity, concentration in the organs is 2.5˜8.0 times of intravenous injection concentration.

[Indications]:

Serves as first-line treatment drug for various solid tumors, and used as first-line plan in combination therapy (EP plan) of etoposide for treating small cell lung cancer (SCLC) or non-small cell lung cancer (NSCLC), and combination (IMP plan) with mitomycin (MMC), oxazaphosphorines ifosfamide (IFO), or vinorelbine (NVB) for non-small cell lung cancer (NSCLC) treatment. Also serves as a major treatment plan in combination chemotherapy when combined mainly with Cisplatin (DDP) for treating advanced ovarian cancer, osteosarcoma and neuroblastoma cell tumors. Combination of adriamycin (ADM) or cefotaxime (CTX) show effective treatment to multiple sites such as squamous-cell carcinoma, moving cancer cells such as head and neck, cervix, esophagus and urinary system tumor. PVB plan (DDP, VLB, BLM) can treat most IV stage nonseminomatous testicular cancer cell with response rate 50˜80%. In addition, the product is a radiation sensitizer, which at abroad is widely used to IV-stage non-small cell lung cancer (NSCLC) in local radiotherapy; enhancing efficacy and improving survival rate.

[Dosage]:

1. General dosage: In accordance to body surface area of 20 mg/m2, the drug is administrated once per day, for 5 consecutive days, or a dosage amount of 30 mg/m2 for 3 consecutive days; requires proper hydration diuretic when used.

2. Large dosage: Dosage amount 80˜120 mg/m2 each time, intravenous infusion, once every 3 to 4 weeks. Maximum dosage should not exceed 120 mg/m2, appropriate dosage at 100 mg/m2. To prevent renal toxicity, sufficient hydration is required: Glucose solution 2000 ml, also with the addition of potassium chloride, mannitol and furosemide 12 hours prior to Cisplatin (DDP) intravenous infusion, or usage of glucose solution; daily urine amount 2000˜3000 ml. During the treatment process keep attention of serum potassium and magnesium changes. Replace with low potassium and magnesium when necessary.

[Adverse drug reaction]:

1. Gastrointestinal: Severe nausea and vomiting as the main limiting toxicity; Acute vomiting usually occurs after 1˜2 hours of administration, and can sustain about one week. As a result, a strong rescue antiemetic is required when using the drug, such as 5- hydroxytryptamine 3 (5-HT3). The receptor when treated with antiemetics such as ondansetron can basically control acute vomiting;

2. Renal toxicity: Dosage-related adverse renal function is the main limiting toxicity. Generally daily dosage exceeding 90 mg/m2 is a risk factor for renal toxicity, mainly causing tubular damage. Acute injury is usually seen 10˜15 days after treatment; blood urea nitrogen (BUN) and creatinine (Cr) increase, and creatinine clearance rate decrease are mostly reversible. Repeated high dosage treatments cause persistent mild to moderate kidney damage. Besides hydration, there is no other methods to prevent renal toxicity;

3. Neurotoxicity: Nerve damages such as auditory nerve damage resulting to tinnitus, and common in hear loss. Peripheral neurotoxicity is related to increase of cumulative dosage, resulting to different degrees of hand, foot sensation loss, sometimes with limb paralysis, trunk muscle weakening, which are generally difficult to restore. Seizures, papilledema or retrobulbar neuritis are less common;

4. Marrow inhibition: Marrow inhibition (white blood cells and /or platelet reduction) are generally rather light, which occurrence is relevant to each dosage; incidence 10˜20% when dosage amount≦100 mg/m2, which incidence about 40% when dosage≧120 mg/m2. However, adverse inhibition is also related with the overlapping of marrow toxicity in combination chemotherapy of other anticancer drugs.

5. Allergy: Swelled face, shortness of breath, tachycardia, low blood pressure, and non-specific maculopapular rash classes may occur;

6. Other: Cardiac dysfunction and liver function changes are rather rare.

[Contraindications]: Renal damaged patients and pregnant women are prohibited of use.

[Notice]:

1. The following patients should be particularly careful with medication: Those with history of renal disease, hematopoietic system dysfunction, acoustic nerve dysfunction, receiving other chemotherapy or radiation treatment before drug medication, and peripheral neuritis induced not by Cisplatin.

2. The following examinations should be conducted before and after treatment, during the treatment period and before each treatment: liver, kidney function, complete blood count, blood calcium and auditory nerve function, and nerve system function. In addition, complete blood count is to be checked during the treatment. The next repeating treatment usually needs await for organ functions restore to normal.

3. Both male and female patients must strictly avoid pregnancy during chemotherapy and after. Genetic counseling is required if pregnancy is desired.

4. Cisplatin may influence focus, driving and abilities of operating machinery.

5. The product should avoid contact with aluminum metal (such as aluminum metal injection needles).

6. The patient must drink enough water during chemotherapy and after.

7. Avoid light during infusion.

[Medication for pregnant and lactating women]:

Pregnant woman is prohibited of use; lactating woman should use with precaution.

[Elder People]:

Glomerular filtration rate and renal plasma flow are reduced with elderly patients, and also reduction in drug excretion. Should use with caution. If renal function is normal, a 70˜90% of the whole dosage amount may be given.

[Drug Interactions]:

(1) When combined with colchicine, probenecid or sulfinpyrazone, Cisplatin might increase the levels of uric acid in the blood. The dosage amount is to be adjusted to control hyperuricemia and gout.

(2) Combination of antihistamines, phenothiazines or thioxanthenes with Cisplatin may mask ototoxic symptoms, such as tinnitus and dizziness.

(3) Cisplatin-induced renal dysfunction can lead to bleomycin (even small dosages) toxicity

(4) Toxicity may increase when used together with various marrow inhibitors or radiation treatment. It is advised to decrease the used amount.

(5) Penicillamine or other chelators will weaken Cisplatin activity. The drug should therefore not be used together chelators.

(6) Proteinuria is increased when used with ifosfamidec, and may also possibly increase ototoxicity.

(7) During Cisplatin chemotherapy, other renal toxic or ototoxic drugs (such as cephalosporins and aminoglycosides) will increase Cisplatin toxicity. Combination needs to be avoided. Diuretics such as furosemide are to be prohibited to increase urine output.

(8) The patient must wait for at least three months to receive virus vaccine after Cisplatin chemotherapy.

[Specification]: Injection: 10/20/30/50 mg injection.

[Storage]: Avoid light. Store in a sealed container at room temperature.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance which can be adapted to the practice of this invention in its various embodiments and equivalents thereof.

What is claimed is:

1. A recombinant human endostatin adenovirus composition for treating cancer, comprising: a therapeutically effective dosage of recombinant human endostatin adenovirus, wherein the composition is suitable for injective administration to a subject in need thereof and wherein the composition is suitable for co-administration with and alleviates toxic side effect of chemotherapy agents while substantially improving the overall effectiveness of treating cancer.

2. The recombinant human endostatin adenovirus composition of claim 1, wherein the human endostatin genes are cloned from human fetal liver cells.

3. The recombinant human endostatin adenovirus composition of claim 1, wherein the recombinant human endostatin adenovirus comprises a type V adenovirus gene and human endostatin gene.

4. The recombinant human endostatin adenovirus composition of claim 1, wherein the cancer is selected from the group consisting of advanced head and neck malignant tumor, soft tissue sarcoma, and advanced breast cancer.

5. The recombinant human endostatin adenovirus composition of claim 1, wherein the chemotherapy agents are selected from Paclitaxel and Cisplatin.

6. The recombinant human endostatin adenovirus composition of claim 1, wherein the composition having a unit dosage is 1.0×10¹² VP.

7. The recombinant human endostatin adenovirus composition of claim 6, wherein the unit dosage of Paclitaxel is160 mg/m² on the third day and the unit dosage of Cisplatin is 25 mg/m² on the third day, the fourth day, and the fifth day of administration of the recombinant human endostatin adenovirus composition.

8. A method for treating cancer, comprising: administering to a subject in need thereof a therapeutically effective amount of recombinant human endostatin adenovirus via a single or multi-point local injective administration applied to a tumor having a size of about 2 cm or greater given once weekly for 2 consecutive weeks as follows: with a medication cycle on the 1st day, a 7-day interval, and a follow-up injection on the eighth day toward the tumor site.

9. The method of claim 8, wherein each injection having a unit dosage of 1.0×1012 VP.

10. The method of claim 9, further comprising administering Paclitaxel at 160 mg/m² on the third day; or Cisplatin 25 mg/m² on the third day, the fourth day, and the fifth day, wherein first-time dosage permitted for adjusting of not exceeding 10%.
